(19) United States
(12) Patent Application Publication
(10) Pub. No.: US 2009/0234227 A1
(43) Pub. Date: Sep. 17, 2009

(54) Ribbed Catheter

(75) Inventor: Karan Punga, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/049,513

(22) Filed: Mar. 17, 2008

(51) Int. Cl. A61B 6/00; A61M 25/00

(52) U.S. Cl. 600/435; 604/264

(57) ABSTRACT

A catheter includes a length sufficient to extend from an entry point into a body to a target site within the body and a substantially circular inner wall defining an inner lumen. The catheter further includes an outer wall separated from and opposing the inner wall, and at least a first rib and a second rib extending from the outer wall. The first rib and second rib define a first fluid flow channel extending along the outer wall.

### TECHNICAL FIELD

The technical field of this disclosure is medical devices, particularly, a catheter.

### BACKGROUND

Catheters are used in a number of medical procedures to deliver medical devices to a target site within a body and other purposes. Catheters, typically, define an inner lumen with an inner wall of the catheter, and this inner lumen can surround dedicated lumens for a number of purposes, such as delivering contrast fluids, delivering devices, or the like. However, with increasing number of dedicated lumens within the catheter, the profile of the catheter increases to accommodate the size of the dedicated lumens.

However, physicians frequently prefer smaller profiles, resulting in a desire to reduce the number of dedicated lumens within the catheter. Frequently, this desire results in catheters failing to include a lumen dedicated to delivering contrast.

It would be desirable to overcome the above disadvantages.

### SUMMARY OF THE INVENTION

One aspect according to the present invention provides a catheter that includes a length sufficient to extend from an entry point into a body to a target site within the body and a substantially circular inner wall defining an inner lumen. The catheter further includes an outer wall separated from and opposing the inner wall, and at least a first rib and a second rib extending from the outer wall. The first rib and second rib define a first fluid flow channel extending along the outer wall.

Another aspect provides a vascular treatment system that includes an introducer sheath and a catheter disposed within the introducer sheath. The catheter includes a length sufficient to extend from an entry point into a body to a target site within the body and a substantially circular inner wall defining an inner lumen. The catheter further includes an outer wall opposing and separated from the inner wall, and at least a first rib and a second rib extending from the outer wall, wherein the first rib and second rib define a first fluid flow channel extending along the outer wall, and wherein the first fluid flow channel extends substantially the length, wherein the first rib and second rib each include a contact surface in frictional contact with an inner surface of the introducer sheath or guide catheter.

Another aspect provides a method of obtaining fluoroscopic images during a medical procedure. The method includes inserting a catheter into a vasculature of a patient, the catheter including a length, and the catheter further including at least one fluid flow channel. The method further includes injecting at least one contrast substance into the fluid flow channel and obtaining a fluoroscopic image of the body lumen surrounding the discharge site of the contrast from the catheter during a medical procedure based on the injection.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIG. 1 is a cross sectional view of a catheter;

FIG. 2 is a cross sectional view of a catheter;

FIG. 3 is a flowchart of a method of obtaining fluoroscopic images during a medical procedure;

FIGS. 4-9 illustrate embodiments of catheter delivery systems;

FIG. 10A is an oblique view of a linear section of one embodiment of the catheter and;

FIGS. 10-13 provide schematic representations illustrating several embodiments of catheters with multiple fluid flow channels, such a the one pictured in FIG. 10A.

### DETAILED DESCRIPTION

Embodiments will now be described by reference to the figures wherein like numbers refer to like structures. The terms “distal” and “proximal” are used herein with reference to the treating clinician during the use of the catheter system: “distal” indicates an apparatus portion distant from, or a direction away from the clinician and “proximal” indicates an apparatus portion near to, or a direction towards the clinician.

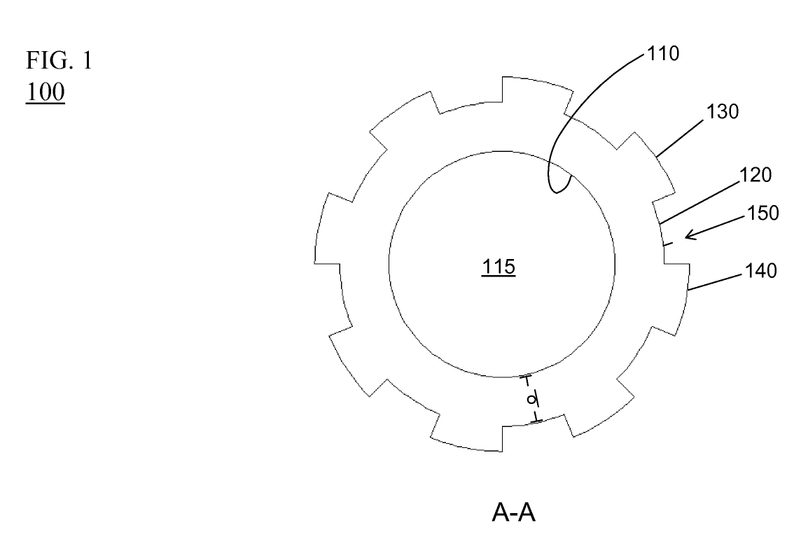

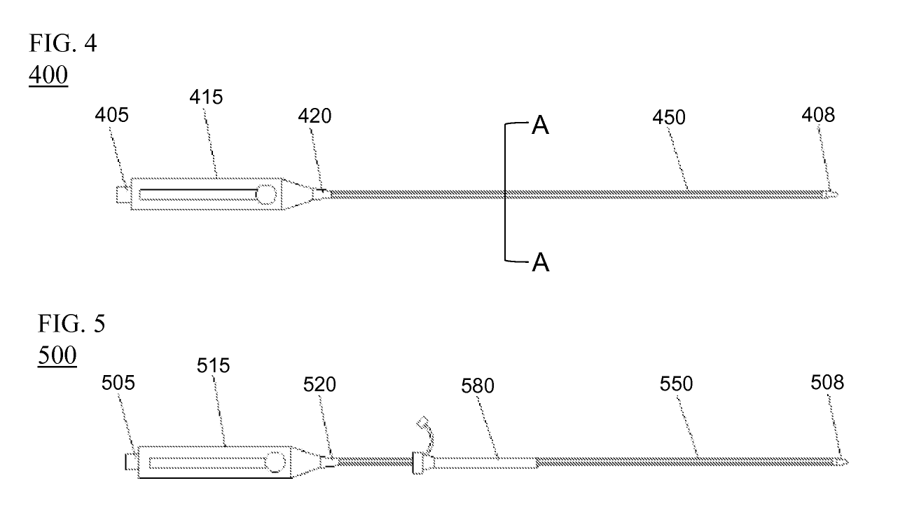

FIG. 1 illustrates one embodiment of a catheter 100, in cross section A-A (FIG. 4). Catheter 100 includes an inner wall 110 that defines inner lumen 115. Any number of devices may be carried, limited only by size of such device and the size of inner lumen 115, within inner lumen 115. In one embodiment, inner wall 110 is substantially circular. In other embodiments, inner wall 110 can be a substantially non-circular tubular shape such as ovoid. Additionally, catheter 100 includes an outer wall 120. Outer wall 120 opposes inner wall 110, and is separated from the inner wall 110 by a wall thickness, d, of the catheter. While not shown in cross sectional FIG. 1, but as seen in FIG. 4, catheter 100 includes a length sufficient to extend from an entry point into a body to a target site within the body. In one embodiment, catheter 100 is a vascular catheter. In other embodiments, catheter 100 is another catheter, such as a urethral catheter, Foley catheter or the like. In one embodiment, catheter 100 serves as a delivery device for a stent (not shown), including drug coated stents. Additionally, catheter 100 includes at least a first rib 130 and a second rib 140 extending radially from the outer wall. The first rib 130 and second rib 140 define a first fluid flow channel 150 extending along the outer wall 120 between the ribs and any element that would span the gap between the top of the ribs. The first fluid flow channel 150 extends substantially the length of catheter 100. Any number of ribs can be included, limited only by the circumference of the catheter and the circumferential thickness of the individual ribs.

The outer surface of first rib 130 and second rib 140 may contact any introducer sheath or guide catheter used in the procedure, and may be near a body lumen wall, such as a vessel wall or urethral wall. Because of such possible contact, the outer surfaces are preferably rounded with few angles (sharp corners). The fluid flow channel 150 extends substantially the entire length of the catheter 100, providing a fluid flow channel for receiving the injection of contrast media during the procedure, but without a dedicated intra-catheter carrying tube. Although illustrated with substantially perpendicular angles, in implementation most surfaces will be radiused.

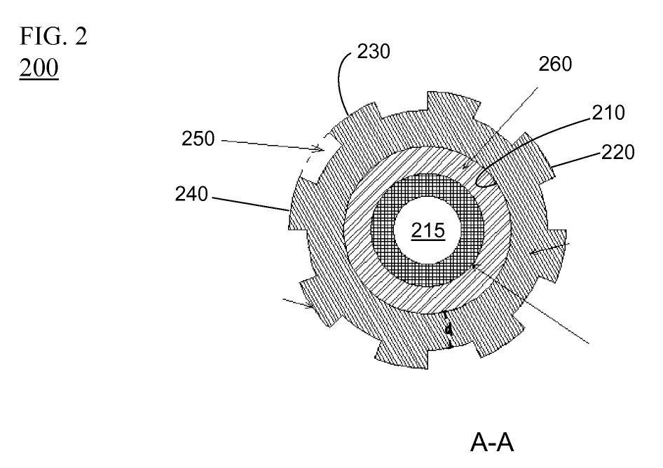

FIG. 2 illustrates another embodiment of a catheter 200 that includes an inner wall 210 that defines inner lumen 215. Any number of devices may be carried within inner lumen 215, limited only by size of such device and the size of inner lumen 215. In one embodiment, inner wall 210 is substantially circular. In other embodiments, inner wall 210 is substantially ovoid. Additionally, catheter 200 includes an outer wall 220. Outer wall 220 opposes inner wall 210, and is separated from the inner wall 210 by a thickness, d, of the catheter. While not shown in cross sectional FIG. 2, catheter 200 has a length sufficient to extend from an entry point into a body to a target site within the body. In one embodiment, catheter 200 is a vascular catheter. In other embodiments, catheter 200 is any other catheter, such as a urethral catheter, Foley catheter or the like. Additionally, catheter 200 includes at least a first rib 230 and a second rib 240 extending radially from the outer wall. The first rib 230 and second rib 240 define a first fluid flow channel 250 extending along the outer wall 220. The first fluid flow channel 250 extends substantially the length of catheter 200. Additionally, catheter 200 includes a middle member 260 disposed within the lumen 215

Catheter 100 and catheter 200 can be designed for a particular application. In one embodiment, the first fluid flow channel is substantially axial. In such embodiments, the axial fluid flow channel runs substantially the entire length of the catheter while reducing any radial portion of the fluid flow channel. Once contrast is injected into the fluid flow channels, the longitudinal or axial fluid flow channels will appear bright fluroscopically. Other embodiments of catheters of FIG. 1 and/or FIG. 2, respectively, 100 and/or 200 include a fluid flow channel that extends axially and radially in a helical pattern along the length of the catheter. Such embodiments will provide a ‘corkscrew’ view during a procedure when fluoroscopic images are taken, when the contrast fluid has been introduced into the fluid flow channels.

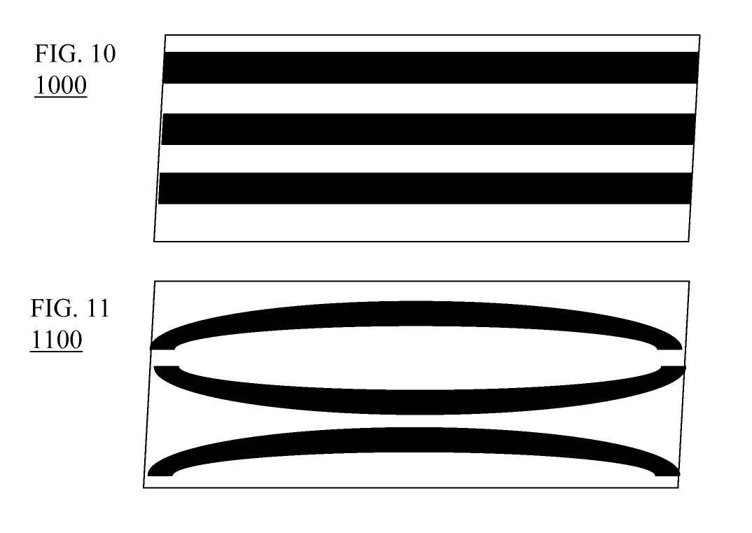

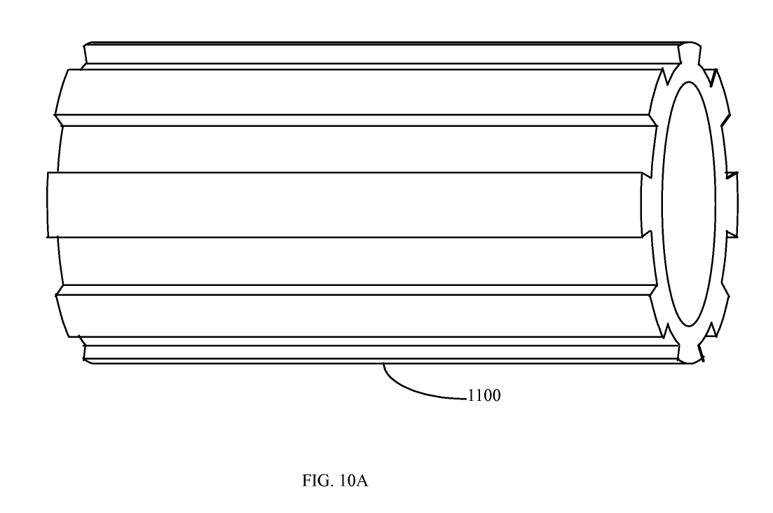

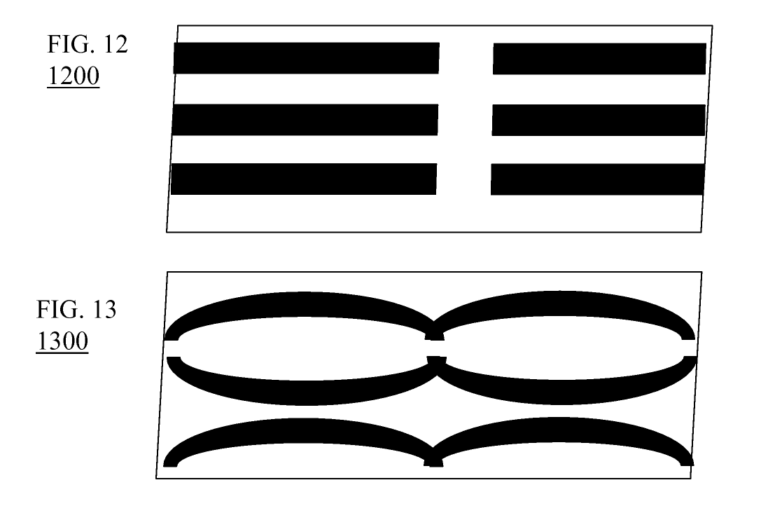

Multiple fluid flow channels are included in various embodiments, such that the catheter includes at least a third rib to define a second fluid flow channel, such as between the third rib and the first rib, or the third rib and the second rib. Any number of ribs may be provided, limited only by the thickness of the ribs, and the outer diameter of the catheter. FIG. 10A is an oblique view of a section of the catheter shaft for example of FIG. 1. FIGS. 10-13 provide a schematic illustration of partial side views of different embodiments of catheters with multiple fluid flow channels. The schematic illustration of FIG. 10 correlates to the catheter shaft section shown in FIG. 10A. The other schematic illustrations presented should be assumed to provide a similar correlation to other possible fluid flow channel configurations similar to that shown in FIG. 10A as will be apparent to those skilled in the art. For example, the fluid flow channels may be substantially parallel (as in FIG. 10) or not substantially parallel (as in FIG. 11). In one embodiment, a first fluid flow channel and a second fluid flow channel intersect such that the first fluid flow channel and second fluid flow channel are in fluidic communication via at least one intersection (as in FIG. 12). In one embodiment, a first intersection and a second intersection are separated by a known distance, such as 6 inches, such that the catheter can function similar to a measuring stick, when imaged fluoroscopically. In one such embodiment, the first fluid flow channel and second fluid flow channel are substantially parallel for a first portion of the length of the catheter and substantially non-parallel for a second portion of the length of the catheter (FIGS. 11 and 13). In yet other embodiments, the fluid flow channels have a substantially consistent width (FIG. 10). In yet other embodiments, the fluid flow channels do not have a substantially consistent width (FIG. 13), and in one such embodiment, the fluid flow channel changes widths at a known separation such that the catheter can function as a measure of distance when imaged fluoroscopically during a procedure. In yet other embodiments, a portion of at least one rib is removed, or has a lower height than elsewhere along the rib, at predetermined distances providing fluidic communication between adjoining fluid flow channels.

Additionally, the height of the ribs may be varied depending on the target site and the diameter of any vasculature or other body vessel to be traversed during the procedure to reduce any discrepancy between the French size of the catheter measured at the outer surface of the ribs and the diameter of the vessel or body lumen to be traversed. In one such embodiment, the first fluid flow channel includes a first marked portion and a second marked portion, and wherein the first marked portion and second marked portion are offset by a predetermined axial distance.

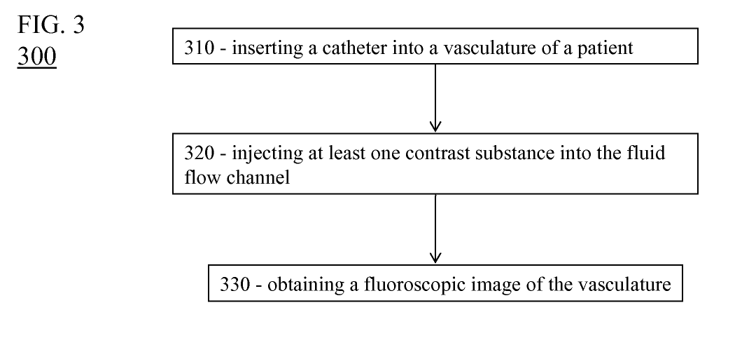

FIG. 3 illustrates one embodiment of a method 300 for obtaining fluoroscopic images during a medical procedure. Method 300 begins at step 310 by inserting a catheter into a vasculature of a patient, the catheter including a length, and the catheter further including at least one fluid flow channel extending substantially the entire length along an outer wall of the catheter. Any appropriate technique for catheter insertion can be, depending on the type of catheter as well as the destination. For example, a urethral catheter is inserted using appropriate urethral insertion techniques, while vascular catheters are inserted using appropriate vascular catheter insertion techniques. During the procedure, at least one contrast substance is injected into at least one fluid flow channel at step 320. The contrast substance may be a contrast dye, or any other substance used to provide improved fluorographic imaging during medical procedures. The contrast substance is housed in a, generally, fluid reservoir disposed outside of the patient's body, and the contrast substance is injected into the fluid flow reservoir, such as with a needle and blocking element (not shown—which prevents backflow of the contrast) placed into fluid communication with the fluid flow reservoir.

At step 330, method 300 obtains fluoroscopic image of the vasculature during a medical procedure based on the injection. Any appropriate fluoroscopy technique can be used. Based on the image, the medial professional undertaking the procedure continues the procedure.

FIG. 4 illustrates a top view of a stent delivery system 400. Stent delivery system 400 is a self expanding stent delivery system without a stability member. Stent delivery system 400 includes a luer fitting 405 at the proximal end of stent delivery system 400 and a tip 408 at the distal end. Handle 415 provides means for gripping and controlling the stent delivery system during a procedure. Additionally, stent delivery system 400 includes a strain relief device 420 at a proximal end of the catheter 450. Catheter 450 is implemented as catheter 100 or catheter 200 and includes at least a first fluid flow channel extending substantially the entire length of the catheter.

FIG. 5 illustrates a top view of a stent delivery system 500. As illustrated, stent delivery system 500 is a self expanding stent delivery system without a stability member, and with an introducer sheath. Stent delivery system 500 includes a luer fitting 505 at the proximal end of stent delivery system 500 and a tip 508 at the distal end. Handle 515 provides means for gripping and controlling the stent delivery system during a procedure. Additionally, stent delivery system 500 includes a strain relief device 520 at a proximal end of the catheter 550. Catheter 550 is implemented as catheter 100 or catheter 200 and includes at least a first fluid flow channel extending substantially the entire length of the catheter. Additionally, introducer sheath 580 assists in introduction of the catheter 500 into the body lumen.

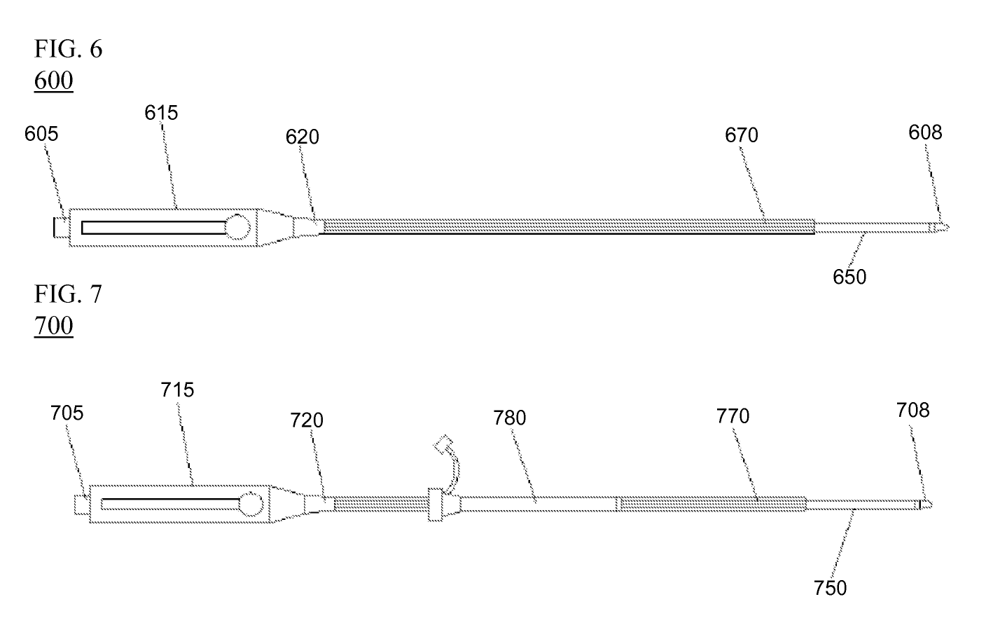

FIG. 6 illustrates a top view of a stent delivery system 600. As illustrated, stent delivery system 600 is a self expanding stent delivery system with a ribbed stability member. In FIG. 6, the stability member 670 includes the fluid flow channels, as outlined with reference to catheter 100 or catheter 200, instead of the catheter 690. Stent delivery system 600 includes a luer fitting 605 at the proximal end of stent delivery system 600 and a tip 608 at the distal end. Handle 615 provides means for gripping and controlling the stent delivery system during a procedure. Additionally, stent delivery system 600 includes a strain relief device 620 at a proximal end of the catheter 650. Catheter 650 is implemented as any catheter known in the art.

FIG. 7 illustrates a top view of a stent delivery system 700. As illustrated, stent delivery system 700 is a self expanding stent delivery system with a ribbed stability member. In FIG. 7, the stability member 770 includes the fluid flow channels, as outlined with reference to catheter 100 or catheter 200, instead of the catheter 750. Stent delivery system 700 includes a luer fitting 705 at the proximal end of stent delivery system 700 and a tip 708 at the distal end. Handle 715 provides means for gripping and controlling the stent delivery system during a procedure. Additionally, stent delivery system 700 includes a strain relief device 720 at a proximal end of the catheter 750. Catheter 750 is implemented as any catheter known in the art. Additionally, introducer sheath 780 assists in introduction of the catheter 750 into the body lumen. Contrast is injected through the introducer sheath and moves into the vasculature or other vessel through the clearance between the outer diameter of the catheter and inner diameter of the introducer sheath. Backward flow is blocked by the hemostasis valve of the introducer. This results in contrast flow in the space between the catheter and introducer sheath/guide catheter.

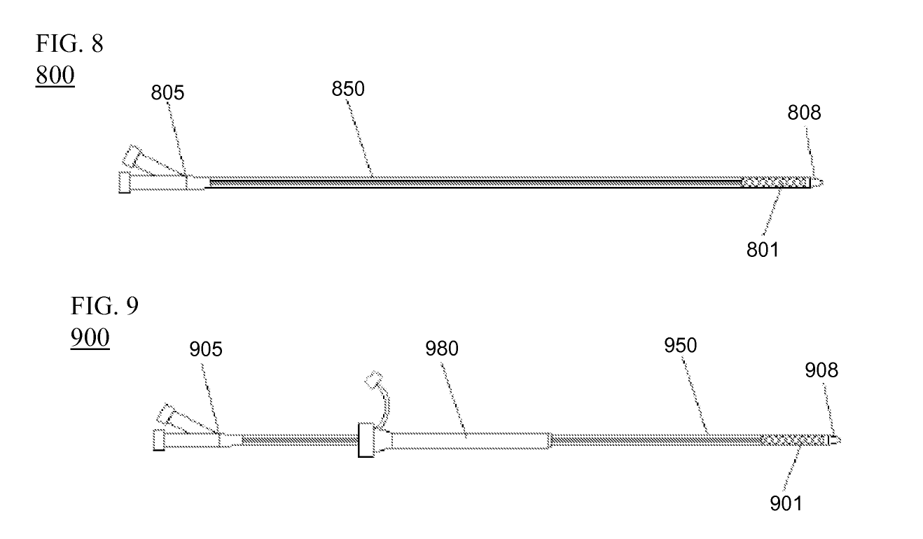

FIG. 8 illustrates a top view of a stent delivery system 800. As illustrated, stent delivery system 800 is a balloon expandable stent delivery system without a stability member. Stent delivery system 800 includes a luer fitting 805 at the proximal end of stent delivery system 800 and a tip 808 at the distal end. Catheter 850 is implemented as catheter 100 or catheter 200 and includes at least a first fluid flow channel extending substantially the entire length of the catheter. Furthermore, FIG. 8 illustrates a stent 801 disposed proximal tip 808 surrounding a balloon (not shown) for stent expansion at a target site.

FIG. 9 illustrates a top view of a stent delivery system 900. As illustrated, stent delivery system 900 is a self expanding stent delivery system without a stability member, and with an introducer sheath. Stent delivery system 900 includes a luer fitting 905 at the proximal end of stent delivery system 900 and a tip 908 at the distal end. Catheter 950 is implemented as catheter 100 or catheter 200 and includes at least a first fluid flow channel extending substantially the entire length of the catheter. Additionally, introducer sheath 980 assists in introduction of the catheter 950 into the body lumen. Furthermore, FIG. 9 illustrates a stent 901 disposed proximal with, and near, tip 908 surrounding a balloon (not shown) for stent expansion at a target site.

Those of skill in the art will recognize that the teachings herein provide for a catheter that provides enhanced means of contrast injection for increased fluoroscopic imaging characteristics of the vasculature, but without a dedicated inner lumen for contrast injection. The fluid flow channels can further be used for estimating internal distances with relative ease during a procedure. By eliminating the need for an internal lumen, the effective French size of the overall catheter is reduced, improving its handling within the body. The catheters disclosed herein are made of any appropriate material, such as the materials generally used to manufacture catheters. Such materials are preferably biocompatible, with sufficient softness as to reduce trauma to the vessel walls and sufficient rigidity as to navigate the vessels.

While specific embodiments are disclosed herein, various changes and modifications can be made without departing from the spirit and scope of the invention.

1. A catheter comprising:
a catheter body having a length sufficient to extend from an entry point into a body to a target site within the body, wherein the catheter body includes an inner wall defining an inner lumen, and an outer wall opposing the inner wall, the outer wall separated from the inner wall, and
at least a first rib and a second rib extending from the outer wall, wherein the first rib and second rib define a first fluid flow channel extending along the outer wall.

2. The catheter of claim 1 wherein the first fluid flow channel is substantially axial.

3. The catheter of claim 1 wherein the first fluid flow channel extends in a helical pattern along the length of the catheter.

4. The catheter of claim 1 wherein the first rib and second rib are integral to the outer wall.

5. The catheter of claim 1 wherein catheter includes at least a third rib, and wherein the third rib defines a second fluid flow channel.

6. The catheter of claim 5 wherein the first fluid flow channel and second fluid flow channel are substantially parallel.

7. The catheter of claim 5 wherein the first fluid flow channel and second fluid flow channel are substantially parallel for a first portion of the length and substantially non-parallel for a second portion of the length.

8. The catheter of claim 1 wherein the first fluid flow channel comprises a substantially consistent width.

9. The catheter of claim 1 wherein the first fluid flow channel includes a first marked portion and a second marked portion, and wherein the first marked portion and second marked portion are offset by a predetermined axial distance.

10. The catheter of claim 1, wherein the first fluid flow channel extends substantially the length of the catheter body.

11. A vascular treatment system comprising:
an introducer sheath; and
a catheter disposed within the introducer sheath, the catheter comprising:
a length sufficient to extend from an entry point into a body to a target site within the body;
a substantially circular inner wall defining an inner lumen;
an outer wall opposing the inner wall, the outer wall separated from the inner wall, and
at least a first rib and a second rib extending from the outer wall, wherein the first rib and second rib define a first fluid flow channel extending along the outer wall, and wherein the first fluid flow channel extends substantially the length, wherein the first rib and second rib each include a contact surface in frictional contact with an inner surface of the introducer sheath.

12. The system of claim 11 wherein the first fluid flow channel is substantially axial.

13. The system of claim 11 wherein the first fluid flow channel extends axially and radially in a helical pattern along the length of the catheter.

14. The system of claim 11 wherein the first rib and second rib are integral to the outer wall.

15. The system of claim 11 wherein catheter includes at least a third rib, and wherein the third rib defines a second fluid flow channel.

16. The system of claim 15 wherein the first fluid flow channel and second fluid flow channel are substantially parallel.

17. The system of claim 15 wherein the first fluid flow channel and second fluid flow channel are substantially parallel for a first portion of the length and substantially non-parallel for a second portion of the length.

18. The system of claim 11 wherein the first fluid flow channel includes a first marked portion and a second marked portion, and wherein the first marked portion and second marked portion are offset by a predetermined axial distance.

19. A method of obtaining fluoroscopic images during a medical procedure, the method comprising:
providing a catheter, the catheter including a length, and the catheter further including at least one fluid flow channel extending substantially the entire length along an outer wall of the catheter;
inserting the catheter into a vasculature of a patient
injecting at least one contrast substance into the fluid flow channel; and
obtaining a fluoroscopic image of a vasculature during the medical procedure based on the injection.
